(19) United States
(12) Patent Application Publication
(10) Pub. No.: US 2008/0207674 A1
(43) Pub. Date: Aug. 28, 2008

(54) Immune Response Modifier Formulations And Methods

(75) Inventors: James D. Stoesz, Inver Grove Heights, MN (US); Cynthia A. Guy, Osseo, MN (US)

Correspondence Address: WOLF GREENFIELD & SACKS, P.C., 600 ATLANTIC AVENUE, BOSTON, MA, 02210-2206, US

(73) Assignee: Coley Pharmaceutical Group, Inc., Wellesley, MA (US)

(21) Appl. No.: 11/722,288

(22) PCT Filed: Dec. 28, 2005
(86) PCT No.: PCT/US05/47374

§ 371 Date: Jun. 20, 2007

Related U.S. Application Data
(60) Provisional application No. 60/640,873, filed on Dec. 30, 2004.

(51) Int. Cl. A61K 31/47

(52) U.S. Cl. 514/293

(57) ABSTRACT

An aqueous parenteral pharmaceutical formulation of the IRM drug compound N-[4-(4-amino-2-ethyl-1H-imidazo[4,5-c]quinolin-1-yl)butyl]methanesulfonamide dissolved in water, buffer selected from citric acid, acetic acid, lactic acid, succinic acid, and tartaric acid, and optionally a tonicity adjuster, preferably selected from sorbitol and mannitol, wherein the pH is no greater than 6 and the formulation is sterile and preferably substantially free of sodium chloride.

### CROSS-REFERENCE TO RELATED APPLICATIONS

This application claims priority to U.S. provisional application 60/640,873, filed Dec. 30, 2004, the entire contents of which is hereby incorporated by reference.

### FIELD OF THE INVENTION

The present invention relates to pharmaceutical formulations and methods related to immune response modifier compounds.

### BACKGROUND

There have been important advances in recent years regarding understanding of the immune system and discovery of drug compounds for modifying immune response to treat or prevent disease. Such immune response modifier (“IRM”) compounds have been discovered in a variety of compound classes, including imidazoquinoline amines, imidazopyridine amines, 6,7-fused cycloalkylimidazopyridine amines, 1,2-bridged imidazoquinoline amines, thiazoloquinoline amines, oxazoloquinoline amines, thiazolopyridine amines, oxazolopyridine amines, imidazonaphthyridine amines, imidazotetrahydronaphthyridine amines, and thiazolonaphthyridine amines. See, for example, U.S. Pat. Nos. 4,689,338; 4,929,624; 5,266,575; 5,268,376; 5,346,905; 5,352,784; 5,389,640; 5,446,153; 5,482,936; 5,756,747; 6,110,929; 6,194,425; 6,331,539; 6,376,669; 6,451,810; 6,525,064; 6,541,485; 6,545,016; 6,545,017; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; 6,756,382; U.S. Patent Publication Nos. 2004/0091491; 2004/0132766; 2004/0147543; and 2004/0176367; and International Patent Application No. PCT/US04/28021 filed on Aug. 27, 2004. Many of these compounds have demonstrated potent immunostimulating, antiviral and antitumor (including anticancer) activity, and have also been shown to be useful as vaccine adjuvants and treatment of TH2-mediated diseases.

However, the ability to provide desired therapeutic benefits of such compounds depends on a variety of factors, including the extent to which they can be formulated and delivered in way that is suitable for particular treatments. Accordingly, there is a need for new methods and formulations to provide the potential therapeutic benefits from these important immunomodifying drug compounds.

### SUMMARY

It is believed that many diseases may be treated by systemic delivery of immune response modifying compounds injection. Unfortunately, however, many such compounds are for one reason or another not well suited for systemic delivery via injection, in some cases due to difficulty in making stable, sterilized formulations suitable for injection having sufficient dissolved drug concentration, low irritation, and that is non-hemolytic.

It has now been found that pharmaceutical formulations especially suitable for injection can be made using the immune response modifier drug compound N-[4-(4-amino-2-ethyl-1H-imidazo[4,5-c]quinolin-1-yl)butyl]methanesulfonamide as an aqueous solution with a buffer selected from the group consisting of citric acid; acetic acid, lactic acid, succinic acid, and tartaric acid, and optionally a tonicity adjuster, preferably selected from the group consisting of sorbitol and mannitol, wherein the pH is no greater than 6. Importantly, these formulations of N-[4-(4-amino-2-ethyl-1H-imidazo[4,5-c]quinolin-1-yl)butyl]methanesulfonamide are sufficiently heat stable to undergo autoclave sterilization. They are also sufficiently stable during storage conditions to permit an extended shelf-life of at least 6 months and generally longer (e.g., 1 to 2 years or longer). The formulations have a pH no greater than 6 and are also, unlike most formulations for injection, preferably substantially free of sodium chloride. A pH higher than 6 appears to enhance degradation, and it has been found that presence of sodium chloride reduces solubility of the drug, apparently due to salt formation.

Although many IRMs may be deliverable in principle via injection, the ability to deliver an IRM compound, in this case a potent toll-like receptor 7 (TLR7) agonist, systemically in a safe, effective, and stable formulation via injection is an important advance in IRM therapy. Formulations of the invention have demonstrated some highly desirable therapeutic results in initial testing for treatment of, e.g., cancer.

Accordingly, the present invention provides, among other things, an aqueous pharmaceutical formulation suitable for injection, comprising the drug compound N-[4-(4-amino-2-ethyl-1H-imidazo[4,5-c]quinolin-1-yl)butyl]methanesulfonamide fully dissolved in a formulation including water, buffer selected from the group consisting of citric acid, acetic acid, lactic acid, succinic acid, and tartaric acid; and optionally a tonicity adjuster, preferably selected from the group consisting of sorbitol and mannitol; wherein the pH is no greater than 6 and the formulation is sterile. The formulation is also preferably substantially free of sodium chloride.

The N-[4-(4-amino-2-ethyl-1H-imidazo[4,5-c]quinolin-1-yl)butyl]methanesulfonamide is generally present at a lower concentration range of at least 1 mg/ml, usually at least 2 mg/ml, in some cases at least 5 mg/ml, and generally less than 16 mg/ml, usually less than 10 mg/ml, and often less than 6 mg/ml. The formulations of the invention surprisingly permit formulation at concentrations higher than would be expected, e.g., above 10 mg/ml.

Also, unlike aqueous nasal spray formulations (WO2005/016275), the present formulations are substantially free of carboxymethylcellulose, and nasal spray formulations do not need to be sterilizable.

Buffer in the formulation may be selected from citric acid, acetic acid, lactic acid, succinic acid, and tartaric acid, with citric acid and/or acetic acid being preferred. For example, the formulation may include a citric acid (and citrate) buffer system. Combinations of buffers can be used, and the buffer(s) can also function as tonicity adjusting agents. The pH is preferably about 5. Also, the pH may be further adjusted as desired by addition of, e.g., sodium hydroxide to the formulation.

A tonicity adjuster may also be used and is preferably selected from the group consisting of sorbitol and mannitol. Tonicity adjuster is optional, particularly when there is already a high buffer concentration, although inclusion of mannitol is generally preferred, as formulations with sorbitol tended to be undergo yellowing under autoclave durations and temperatures (e.g., 99 minutes at 136° C.) and with increasing pH.

Also provided are methods of delivering N-[4-(4-amino-2-ethyl-1H-imidazo[4,5-c]quinolin-1-yl)butyl]methanesulfonamide by injecting any of the above formulations intraveneously, subcutaneously, intramuscularly, or into a selected tissue site, such as into a tumor mass. Injection can be by syringe, intravenous catheter, or any other such invasive delivery system, all of which will normally require a sterile formulation. Formulations suitable for subcutaneous injection, as well as IV and other forms of injection, preferably have a pH of about 5 and include citric acid or acetic acid buffer and mannitol tonicity adjuster.

The terms “comprises” and variations thereof do not have a limiting meaning where these terms appear in the description and claims.

The term “solution” refers to a combination of two or more substances uniformly dispersed throughout a single phase, so that the combination is homogeneous at the molecular or ionic level.

The term “substantially free” is used to indicate that the amount present in the composition or formulation is below the level that causes any material impact on the formulation characteristics, e.g., in terms of solubility, viscosity or degradation. Thus, a formulation including trace amounts of a compound may still be considered to be substantially free of such compound.

As used herein, “a,” “an,” “the,” “at least one,” and “one or more” are used interchangeably. Thus, for example, an aqueous gel that comprises “a” preservative can be interpreted to mean that the gel includes “one or more” preservatives.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### DETAILED DESCRIPTION OF ILLUSTRATIVE EMBODIMENTS

Sterile injectable formulations of N-[4-(4-amino-2-ethyl-1H-imidazo[4,5-c]quinolin-1-yl)butyl]methanesulfonamide disclosed herein can include a range of drug concentrations, with lower limits based on minimum therapeutic potency of the drug and upper limits based primarily on solubility of the drug. In general, the concentration of drug will be from about 1 to 16 mg/ml (or about 0.1% to 1.6% by weight), often between 1 and 5 mg/ml, although higher concentrations may be desired (e.g., for subcutaneous delivery) so a concentration of at least 2 mg/ml, and in some cases at least 5 mg/ml. may be desired.

The injection may be, e.g., intravenous, subcutaneous, intramuscular, or into a selected tissue site, such as into a tumor mass. The formulations are preferably stable under autoclave sterilization conditions, are photostable, are sufficiently stable during long term storage conditions to provide a shelf life of at least 6 months and preferably 1-2, or more, years, are relatively non-irritating remain in solution when injected, and are non-hemolytic.

The amount of N-[4-(4-amino-2-ethyl-1H-imidazo[4,5-c]quinolin-1-yl)butyl]methanesulfonamide compound to be dosed that will be therapeutically effective in a specific situation will depend on such things as the size and immune system function of the individual being treated, the dosing regimen, the application site, the particular formulation, and the condition being treated. As such, it is generally not practical to identify specific administration amounts herein; however, those skilled in the art will be able to determine appropriate therapeutically effective amounts based on the guidance provided herein, information available in the art pertaining to IRM compounds, and routine testing. The term “a therapeutically effective amount” thus means an amount of the IRM compound sufficient to induce a therapeutic or prophylactic effect, such as cytokine induction, inhibition of TH2 immune response, antiviral or antitumor activity, reduction of scarring, or enhanced wound healing.

An amount of formulation at a given drug concentration effective to induce cytokine biosynthesis is an amount sufficient to cause one or more cell types, such as monocytes, macrophages, dendritic cells and B-cells to produce an amount of one or more cytokines such as, for example, IFN-α, TNF-α, IL-1, IL-6, IL-10 and IL-12 that is increased (induced) over a background level of such cytokines. The precise amount will vary according to factors known in the art but is expected to be an amount so as to deliver N-[4-(4-amino-2-ethyl-1H-imidazo[4,5-c]quinolin-1-yl)butyl]methanesulfonamide at a dose of about 100 ng/kg to about 50 mg/kg, preferably about 1 μg/kg to about 5 mg/kg.

### EXAMPLES

Objects and advantages of this invention are further illustrated by the following examples, but the particular materials and amounts thereof recited in these examples, as well as other conditions and details, should not be construed to unduly limit this invention. Unless stated otherwise, all percentages are by weight based on weight of the final formulation.

The IRM used in the examples is N-[4-(4-amino-2-ethyl-1H-imidazo[4,5-c]quinolin-1-yl)butyl]methanesulfonamide, which is a sulfonamide substituted imidazoquinoline amine, the synthesis of which is described, for example, in U.S. Pat. No. 6,677,349, Example 236.

### Excipients

The excipients that were used to prepare the formulations are shown in Table 1 below.

TABLE 1

Sterile water for injection, USP
L-lactic acid,
1M L-lactic acid
L-tartaric acid
Acetic acid
Citric acid
Sorbitol
Mannitol
1N Sodium hydroxide (1N NaOH)
10N Sodium hydroxide (10N NaOH)
USP United States Pharmacopeia

### Preparation of the Formulations

The formulations were prepared using the following general method. The buffer was mixed with water. The N-[4-(4-amino-2-ethyl-1H-imidazo[4,5-c]quinolin-1-yl)butyl]methanesulfonamide was added and stirred until dissolved. The resulting solution was mixed with additional water and a tonicity agent as necessary. The amount of the tonicity agent added was determined by calculating the osmolarity of the buffer on an Osmette osmometer (Precisions Systems Inc., Natick, Mass.), then calculating the amount of tonicity agent needed to make up the difference. A pH adjuster was added, as necessary, to adjust each formulation to the desired pH. Finally, water was added to each formulation to adjust to the final formulation weight and the formulations were filtered. Formulations prepared by this method can be found in Tables 2 through 5 below. Formulations in which a precipitate formed were placed in a 25° C. water bath for one week to allow them to equilibrate prior to filtering.

### Stability Test Method

Formulations of the invention were tested for degradation of the formulations by measuring the impurity N-[4-(2-ethyl-4-oxo-4,5-dihydro-1H-imidazo[4,5-c]quinolin-1-yl)butyl]methanesulfonamide and the color change in the formulations after degradation of the formulations using the following test method.

The formulations were placed in an autoclave for 99 minutes at 136° C. The sterilized formulations were then removed from the autoclave and visually observed for discoloration and measured using HPLC for the impurity N-[4-(2-ethyl-4-oxo-4,5-dihydro-1H-imidazo[4,5-c]quinolin-1-yl)butyl]methanesulfonamide. The impurity was measured as a percentage (% 4-keto) of the N-[4-(4-amino-2-ethyl-1H-imidazo[4,5-c]quinolin-1-yl)butyl]methanesulfonamide in the formulation.

### Examples 1-29

A series of aqueous formulations containing N-[4-(4-amino-2-ethyl-1H-imidazo[4,5-c]quinolin-1-yl)butyl]methanesulfonamide were prepared and tested for degradation using the test method described above. Tables 2 through 5 show the composition of each formulation and the test result.

TABLE 2

| | Formulations (percentage weight by weight) | | | | | | | |
|---|---|---|---|---|---|---|---|---|
| | | | | 0.03 M | | | | |
| | 0.1M Acetate | | | Acetate | 0.02M Acetate | | 0.1M Citrate | |
| Ingredients | Ex 1 | Ex 2 | Ex 3⁺ | Ex 4 | Ex 5 | Ex 6 | Ex 7 | Ex 8 |
| IRM | 1.0 | 1.0 | 1.0 | 1.0 | 1.0 | 1.0 | 1.0 | 1.0 |
| Acetic acid | 0.6 | 0.6 | 0.6 | 0.18 | 0.12 | 0.12 | — | — |
| Citric acid | — | — | — | — | — | — | 2.1 | 2.1 |
| Sorbitol | 2 | 2 | 2 | 5 | 5 | 5 | 0 | 0 |
| 1N NaOH | 0 | 4.2 | 1.9* | 0 | 0 | drops | 8.3 | 15.7 |
| Water | qs | qs | qs | qs | qs | qs | qs | qs |
| pH | 4.2 | 5.0 | 6.0 | 3.9 | 5.0 | 6.0 | 4.0 | 5.0 |
| % 4-keto | 0.5 | 1.2 | 1.5 | 0.4 | 0.8 | 1.2 | 0.3 | 0.9 |
| Color | none | orange | orange | orange | orange | orange | none | none |

*Some 10N NaOH was used to keep the volume of the base low.
⁺Precipitated

TABLE 3

| | Formulations (percentage weight by weight) | | | | | | | |
|---|---|---|---|---|---|---|---|---|
| | 0.1M | | | | | | | 0.04M |
| | Citrate | 0.02M Citrate | | | 0.1M Lactate | | | Lactate |
| Ingredients | Ex 9 | Ex 10 | Ex 11 | Ex 12 | Ex 13 | Ex 14 | Ex 15⁺ | Ex 16 |
| IRM | 1.0 | 1.0 | 1.0 | 1.0 | 1.0 | 1.0 | 1.0 | 1.0 |
| Citric acid | 2.1 | 0.42 | 0.42 | 0.42 | — | — | — | — |
| L-lactic acid | — | — | — | — | 1.0 | 1.0 | 1.0 | — |
| 1M L-lactic | — | — | — | — | — | — | — | 2.0 |
| acid | | | | | | | | |
| Sorbitol | 0 | 5 | 5 | 5 | 2 | 2 | 2 | 5 |
| 1N NaOH | 9.7* | NA | drops | 0.7 | 2.8 | 3.1 | 6.2 | NA |
| Water | qs | qs | qs | qs | qs | qs | qs | qs |
| pH | 6.0 | 4.0 | 5.0 | 6.0 | 4.0 | 5.4 | 6.2 | 4.0 |
| % 4-keto | 1.9 | 0.3 | 0.9 | 1.2 | 0.3 | 0.5 | 0.6 | 0.2 |
| Color | none | none | yellow | yellow | none | none | none | none |

*Some 10N NaOH was used to keep the volume of the base low.
⁺Precipitated

TABLE 4

| | Formulations (percentage weight by weight) | | | | | | | |
|---|---|---|---|---|---|---|---|---|
| | 0.04M Lactate | | 0.1M Tartrate | | | 0.02 Tartrate | | |
| Ingredients | Ex 17 | Ex 18⁺ | Ex 19 | Ex 20 | Ex 21⁺ | Ex 22 | Ex 23 | Ex 24⁺ |
| IRM | 1.0 | 1.0 | 1.0 | 1.0 | 1.0 | 1.0 | 1.0 | 1.0 |
| L-tartaric | — | — | 1.5 | 1.5 | 1.5 | 0.3 | 0.3 | 0.3 |
| acid | | | | | | | | |
| 1M L-lactic | 2.0 | 2.0 | — | — | — | — | — | — |
| acid | | | | | | | | |
| Sorbitol | 5 | 5 | 0 | 0 | 0 | 5 | 5 | 5 |
| 1N NaOH | drops | drops | 11.0 | 1.6 | 3.2* | drops | 2.2 | 8.9 |
| Water | qs | qs | qs | qs | qs | qs | qs | qs |
| pH | 5.1 | 6.3 | 4.0 | 5.1 | 6.2 | 4.0 | 5.2 | 6.2 |
| % 4-keto | 0.4 | 0.6 | 0.3 | 0.7 | 1.3 | 0.3 | 0.6 | 0.7 |
| Color | yellow | yellow | none | none | none | yellow | orange | orange |

*Some 10N NaOH was used to keep the volume of the base low.
⁺Precipitated

TABLE 5

| | Formulations (percentage weight by weight) | | | | |
|---|---|---|---|---|---|
| | | | | 0.03M | 0.05 M |
| | 0.02M Citrate | | | Citrate | Acetate |
| Ingredients | Ex 25 | Ex 26 | Ex 27 | Ex 28 | Ex 29 |
| IRM | 0.15 | 0.2 | 0.4 | 0.15 | 0.15 |
| Citric acid | 0.42 | 0.42 | 0.42 | 0.63 | — |
| Acetic Acid | — | — | — | — | 0.3 |
| Mannitol | 4.5 | 4.5 | 4.5 | 4.0 | 4.0 |
| 1N NaOH | 3.9 | 3.8 | 3.8 | 6.0 | 3.5 |
| Water | qs | qs | qs | qs | qs |
| pH | 5.0 | 5.0 | 5.0 | 5.0 | 5.0 |
| % 4-keto | 0.7 | — | — | — | 0.9 |
| Color | none | — | — | — | none |

In addition to those listed, other ingredients suitable for an injectable formulation of the invention may also be included. For example, compatible excipients listed by Powell, et al., “Compendium of Excipients for Parenteral Formulations,” Journal of Pharmaceutical Science & Technology, Vol. 52, No. 5, pages 238-311 (September-October 1996) may be included if desired. Some examples believed to be compatible include, but are not limited to, acetate, sodium acetate, ascorbic acid, benzyl alcohol, citrate, mono-, di- and tri-sodium citrate, dextran 40, cyclodextrin disodium edetate (EDTA), ethanol, glucose, glycerin, glycerol, HCl, maleic acid, methyl paraben, propylparaben, potassium phosphate (mono and di basic), sodium phosphate (mono and di basic), polyethylene glycol, phenol, and KCl.

The formulation may also include, or be administered in conjunction with, other active agents useful for treating a given condition, as well as in conjunction with other formulations of N-[4-(4-amino-2-ethyl-1H-imidazo[4,5-c]quinolin-1-yl)butyl]methanesulfonamide (see, e.g., 60/640,873, filed Dec. 30, 2004, related application attorney docket 60330WO003, filed even date herewith, entitled Multi-Route Administration of Immune Response Modifier Compounds). Such additional agents may include, for example, additional immune response modifiers, antivirals, antibiotics, antibodies, proteins, peptides, oligonucleotides, chemotherapeutic agents, cytotoxoid agents, cytokines, vaccines or a tumor necrosis factor receptor (TNFR) agonist.

### Uses

Formulations of the invention induce the production of certain cytokines and are useful as immune response modifiers that can modulate the immune response in a number of different ways, rendering them useful in the treatment of a variety of disorders.

Among other effects, these and other cytokines can inhibit virus production and tumor cell growth, making the formulations useful for, e.g., treatment of viral and neoplastic diseases. It should also be noted that the formulations may be administered prior to acquiring a disease so that administration of the formulation may provide a prophylactic treatment.

In addition to the ability to give rise to cytokine induction, formulations of the invention may bring about an effect on other aspects of the innate immune response. For example, natural killer cell activity may be stimulated, an effect that may be due to cytokine induction. The formulations may also bring about activation of macrophages, which in turn stimulate secretion of nitric oxide and the production of additional cytokines. Further, the formulations may bring about proliferation and differentiation of B-lymphocytes.

Formulations of the invention may also bring about an effect on the acquired immune response. For example, the production of the T helper type 1 (T_{H}1) cytokine IFN-γ may be induced indirectly and the production of the T helper type 2 (T_{H}2) cytokines IL-4, IL-5 and IL-13 may be inhibited upon administration of the formulations.

Examples of conditions for which formulations described herein may be used as treatments include, but are not limited to:

a) viral diseases such as, for example, diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picornavirus (e.g., rhinovirus or enterovirus), an orthomyxovirus (e.g., influenzavirus, including H5N1 avian flu virus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV);

(b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella;

(c) other infectious diseases, such chlamydia, fungal diseases including but not limited to candidiasis, aspergillosis, histoplasmosis, cryptococcal meningitis, or parasitic diseases including but not limited to malaria, pneumocystis camii pneumonia, leishmaniasis, cryptosporidiosis, toxoplasmosis, and trypanosome infection;

(d) neoplastic diseases, such as intraepithelial neoplasias, cervical dysplasia, actinic keratosis, basal cell carcinoma, squamous cell carcinoma, renal cell carcinoma, Kaposi's sarcoma, melanoma, leukemias including but not limited to myelogeous leukemia, acute lymphocytic leukemia, chronic lymphocytic leukemia, multiple myeloma, non-Hodgkin's lymphoma, cutaneous T-cell lymphoma, B-cell lymphoma, and hairy cell leukemia, and other cancers;

(e) T_{H}2-mediated, atopic diseases, such as atopic dermatitis or eczema, eosinophilia, asthma, allergy, allergic rhinitis, and Ommen's syndrome;

(f) certain autoimmune diseases such as systemic lupus erythematosus, essential thrombocythaemia, multiple sclerosis, discoid lupus, alopecia greata; and

(g) diseases associated with wound repair such as, for example, inhibition of keloid formation and other types of scarring, and enhancing wound healing, including chronic wounds, such as those associated with diabetic foot ulcers and the like.

Additionally, formulations of the present invention may be useful as a vaccine adjuvant for use in conjunction with any material that raises either humoral and/or cell mediated immune response, such as, for example, live viral, bacterial, or parasitic immunogens; inactivated viral, tumor-derived, protozoal, organism-derived, fungal, or bacterial immunogens; toxoids; toxins; self-antigens; polysaccharides; proteins; glycoproteins; peptides; cellular vaccines; DNA vaccines; autologous vaccines; recombinant proteins; and the like, for use in connection with, for example, BCG, cholera, plague, typhoid, hepatitis A, hepatitis B, hepatitis C, influenza A, influenza B, parainfluenza, polio, rabies, measles, mumps, rubella, yellow fever, tetanus, diphtheria, hemophilus influenza b, tuberculosis, meningococcal and pneumococcal vaccines, adenovirus, HIV, chicken pox, cytomegalovirus, dengue, feline leukemia, fowl plague, HSV-1 and HSV-2, hog cholera, Japanese encephalitis, respiratory syncytial virus, rotavirus, papilloma virus, yellow fever, and Alzheimer's disease.

Formulations of the present invention may be particularly helpful in individuals having compromised immune function. For example, compounds or salts may be used for treating the opportunistic infections and tumors that occur after suppression of cell mediated immunity in, for example, transplant patients, cancer patients and HIV patients.

The invention thus also provides, for example, a method of treating a viral infection in an animal and a method of treating a neoplastic disease in an animal comprising administering via injection an effective amount of a formulation of the invention to the animal. An amount effective to treat or inhibit a viral infection is an amount that will cause a reduction in one or more of the manifestations of viral infection, such as viral lesions, viral load, rate of virus production, and mortality as compared to untreated control animals. The precise amount that is effective for such treatment will vary according to factors known in the art but is expected to be an amount so as to deliver a N-[4-(4-amino-2-ethyl-1H-imidazo[4,5-c]quinolin-1-yl)butyl]methanesulfonamide dose of about 100 ng/kg to about 50 mg/kg, preferably about 1 μg/kg to about 5 mg/kg. An amount of formulation effective to treat a neoplastic condition is an amount that will cause a reduction in tumor size or in the number of tumor foci. Again, the precise amount will vary according to factors known in the art but is expected to be an amount at a given drug concentration to deliver via injection a N-[4-(4-amino-2-ethyl-1H-imidazo[4,5-c]quinolin-1-yl)butyl]methanesulfonamide dose of about 100 ng/kg to about 50 mg/kg, for example about 1 μg/kg to about 5 mg/kg.

Particular examples of uses of formulations of the invention delivered via injection include, but are not limited to, treatment of metastatic melanoma and chronic lymphocytic leukemia (see, e.g., WO05/023190).

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. An aqueous pharmaceutical formulation suitable for injection, comprising:
the drug compound N-[4-(4-amino-2-ethyl-1H-imidazo[4,5-c]quinolin-1-yl)butyl]methanesulfonamide fully dissolved in the formulation;
water;
buffer selected from the group consisting of citric acid, acetic acid, lactic acid, succinic acid, and tartaric acid; and
optionally a tonicity adjuster;
wherein the pH is no greater than 6 and the formulation is sterile.

2. The formulation of claim 1, wherein the N-[4-(4-amino-2-ethyl-1H-imidazo[4,5-c]quinolin-1-yl)butyl]methanesulfonamide is present at a concentration of at least 1 mg/ml.

3. The formulation of claim 1, wherein the N-[4-(4-amino-2-ethyl-1H-imidazo[4,5-c]quinolin-1-yl)butyl]methanesulfonamide is present at a concentration of at least 2 mg/ml.

4. The formulation of claim 1, wherein the N-[4-(4-amino-2-ethyl-1H-imidazo[4,5-c]quinolin-1-yl)butyl]methanesulfonamide is present at a concentration of at least 5 mg/ml.

5. The formulation of claim 1, wherein the N-[4-(4-amino-2-ethyl-1H-imidazo[4,5-c]quinolin-1-yl)butyl]methanesulfonamide is present at a concentration of at least mg/ml.

6. The formulation of any preceding claim wherein the buffer is selected from the group consisting of citric acid and acetic acid.

7. The formulation of claim 6, wherein the buffer is citric acid.

8. The formulation of any preceding claim, wherein the pH is 5.

9. A method of delivering the drug compound N-[4-(4-amino-2-ethyl-1H-imidazo[4,5-c]quinolin-1-yl)butyl]methanesulfonamide by injecting into a subject a formulation comprising:
N-[4-(4-amino-2-ethyl-1H-imidazo[4,5-c]quinolin-1-yl)butyl]methanesulfonamide fully dissolved in the formulation;
water;
buffer selected from the group consisting of citric acid, acetic acid, lactic acid, succinic acid, and tartaric acid; and
optionally a tonicity adjuster;
wherein the pH is no greater than 6 and the formulation is sterile.

10. The method of claim 9, wherein the formulation is injected intravenously.

11. The method of claim 9, wherein the formulation is injected subcutaneously.

12. The method of claim 9, wherein the formulation is injected into a tumor mass.

13. A method of treating a disease by injecting into a subject in need of treatment of the disease a formulation comprising:
N-[4-(4-amino-2-ethyl-1H-imidazo[4,5-c]quinolin-1-yl)butyl]methanesulfonamide fully dissolved in the formulation;
water;
buffer selected from the group consisting of citric acid, acetic acid, lactic acid, succinic acid, and tartaric acid; and
optionally a tonicity adjuster;
wherein the pH is no greater than 6 and the formulation is sterile.

14. The method of claim 13, wherein the injection is subcutaneous.

15. The method of claim 13, wherein the injection is intraveneous.

16. The method of claim 13, wherein the injection is directly into a tumor mass.

17. The method of claim 13, wherein the disease is metastatic melanoma.

18. The formulation or method of any preceding claim, wherein the formulation includes a tonicity adjuster selected from the group consisting of sorbitol and mannitol.

19. The formulation or method of claim 18, wherein the tonicity adjuster is mannitol.

20. The formulation or method of any preceding claim, wherein the formulation is substantially free of sodium chloride.
